(19) United States
(12) Patent Application Publication
(10) Pub. No.: US 2007/0148227 A1
(43) Pub. Date: Jun. 28, 2007

(54) PHYSICALLY/MOLECULARLY DISTRIBUTED AND/OR CHEMICALLY BOUND MEDICAMENTS IN CAPSULE SHELLS

(75) Inventor: Hemant N. Joshi, Parsippany, NJ (US)

Correspondence Address: Tara Enterprises, 41 Leah Way, Parsippany, NJ, 07054, US

(73) Assignee: Hemant N. Joshi, Parsippany, NJ (US)

(21) Appl. No.: 11/306,398

(22) Filed: Dec. 27, 2005

(51) Int. Cl. A61K 9/48

(52) U.S. Cl. 424/451

(57) ABSTRACT

The present invention proposes a design to incorporate medicaments in the capsule shells (body and cap). Medicaments in the cap and body of the capsules may be different. Other medicaments in the form of granules, beads etc. can be filled in the capsules, which may contain medicaments capsule shell. Thus, the same capsule may contain medicaments in the core matrix and in the shell. The key advantage of incorporation of drug in the shell is to obtain a desired rate of release of the medicament, mainly for potent drugs. Other advantage is to produce a combination drug delivery system. The concept can be used for the hard gelatin, hard non-gelatin, soft gelatin and soft non-gelatin capsules. The type of medicaments can be from any class, but should be low-dose medicaments. The medicament has to be stable in capsule shell during manufacture and during appropriate storage conditions for the capsules.

### BACKGROUND OF THE INVENTION

Combination drug therapy has been gaining a lot of importance in recent times. The reasons could be-avoidance of taking multiple tablets/capsules per day, savings on co-payment for different medicines and assurance of patient compliance to drug therapies. It is important to show that different drugs combined in the same dosage form should be stable during storage and should not interact physically or chemically with other drugs or excipients to produce degradation products. Also, each drug should show the desired release rate from the dosage form to get absorbed in sufficient quantities upon oral administration or release the drug to surrounding environment in case of other delivery routes.

Pharmaceutical capsule dosage forms are widely used in delivering drugs. The capsules are normally prepared using gelatin and other excipients. In recent times, a polymer such as HPMC (hydroxypropylmethyl cellulose) is employed to make capsules. In 2004 patent by Chen et al. (U.S. Pat. No. 6,752,953), authors described the usage of other polymers such as cellulose derivatives, acrylates, polyolefins and vinyl polymers to prepare capsules.

There are mainly two methods to prepare capsules: dip-coating and heat-melting. A liquid mass is by produce by dissolving the capsule compositions in a solvent system or by melting at an appropriate temperature. A pin maintained at a certain temperature dips in these solutions and is withdrawn at a pre-determined rate. The capsules are then dried. The method has been employed to prepare the body and cap of the capsules.

InnerCap, a UK-based company proposed combination capsules in which a capsule may contain another small capsule or a tablet along with granules. The granules may be made up of beads or other forms, which may contain more than one type of drug molecules. This way, more than one type of drug may be combined in the same capsule.

Soft gelatin capsules are another form of capsules in which a liquid matrix is filled. It has created a niche market of its own in the drug delivery technology. In coming times, soft non-gelatin may be introduced in the market due to advent of new polymeric systems.

### SUMMARY OF THE INVENTION

In the present invention proposes a new way of combining more than one drug component in the capsule formulations so that the release rate of each drug can be controlled to desired value. The method comprises: (1) physically dispersing the drug in the capsule shell during the manufacture of the capsules; (2) chemically binding the drug to the capsule component. The capsules may be prepared with gelatin, HPMC and other known polymers. The capsules may be prepared by dip-coating or heat-melting methods.

### BRIEF DESCRIPTION OF DRAWINGS

FIG. 1 shows cap of a capsule containing drug molecules dispersed. The dispersion of molecules might be physical or via chemical-binding.

### DETAILED DESCRIPTION

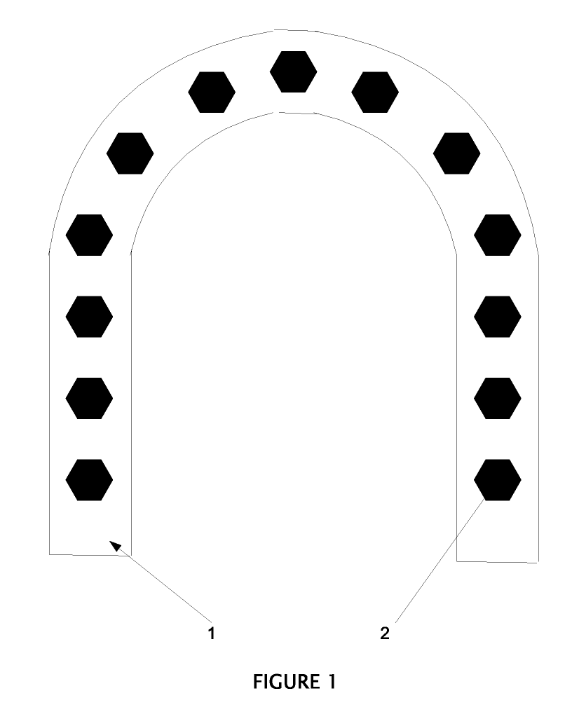

FIG. 1 depicts the capsule cap 1 and drug molecules 2. A similar structure can be shown for the capsule body. The composition of the capsule body or the cap may consist of gelatin, HPMC, cellulose-derivatives, acrylates, polyolefins, vinyl polymers and other polymeric systems. The main constituent of the capsule shell may be a combination of polymers mentioned above. The shell may also contain other excipients such as plasticizers, emulsifiers, stabilizers, colorants etc. The rate of capsule dissolution in a desired media may be altered using selected combination of the capsule composition.

The medicament in the capsules 2 can be physically or molecularly dispersed. The medicament 2 in the powder form can be physically dispersed in the capsule composition. The medicament 2 may be dissolved in the capsule composition to disperse it at a molecular level. The medicament may form an ion-pair bond with the groups in the polymer or the excipients used to prepare the capsule composition. The medicament 2 may form a chemical bond with the polymer or the excipients used to prepare the capsule composition. The chemical bond can be of any nature—peptide, an ester or other kinds.

Bioavailability of a drug constitutes of two features—the rate and the extent of absorption. For drugs with narrow therapeutic indices, it is critical to maintain appropriate drug levels in the blood or tissues. For potent drugs, one must avoid dumping of drugs in a short period from the delivery device into the gastro-intestinal tract so that one can avoid erratic blood levels for the medicament.

The amount of medicament 2 which can dispersed in the capsule shell (body and/or cap) is limited due to low capsule weights. Also, the addition of drug to the capsule composition may alter the capsule shell properties. Thus, only potent drugs can be dispersed in the capsule shell composition. Any type of drug can be included in the capsule shell composition.

The release of drug from the capsule shell can be controlled by three mechanisms-diffusion through the shell matrix, hydrolysis of medicament-polymer chemical bond or the combination of both.

Other medicaments may be placed in the capsule core in the form of granules, beads, tablets, capsules etc.

It is critical to establish the stability of medicament 2 dispersed in the capsule shell (body and cap) during manufacture and storage of capsules. It is important to establish the desired release rate of medicament 2 from the capsule shell under pre-determined conditions such as in the acidic/basic media, in the presence of bile acids/food.

What is claimed is:

1. A capsule delivery system in which the medicament/medicaments are dispersed in the shell (body and cap) so that one can control the release of medicament.

2. The design of capsule shell comprising of medicament or medicaments, which releases the drug at a desired rate. The capsule shell will produce a form of a controlled-release drug delivery system. The release or medicament could be immediate or sustained/slow.

3. The medicament in claim 1 may be molecularly or physically dispersed in the capsule shell.

4. The medicament in claim 1 may be chemically bound to the capsule components. Chemical binding is defined as covalent or ion-pair binding.

5. The material used in the capsule shell formulation in claim 1 could be gelatin, HPMC, other cellulose derivatives, vinyl, acrylates, polyolefins or their combinations.

6. The capsule shell comprising of medicament from claim 1 can be used for the administration of drug by oral, implant, intra-uterine, vaginal routes. Implants may be placed in any body cavities such as subcutaneous, abdominal, in or around tumors etc.

7. The capsule shell comprising of medicament from claim 1, where the drug is chemically stable during manufacture of the capsule and under appropriate storage conditions.

8. The capsule shell comprising of medicament from claim 1 from which the drug is released by diffusion controlled, hydrolysis controlled (of the drug-polymer bond) and/or combination of both methods. The release of drug can be influenced by other external force such as enzyme needed for the polymer degradation or water for generating pores upon swelling.

9. The methodology of embedding medicament in the capsule shell as in claim 1 can be used for the hard gelatin, hard non-gelatin, soft gelatin and soft non-gelatin capsules.

10. One or more medicament 2 can be incorporated in the shell (body and/or cap) either physically or chemically or by combination of the two methods.
